(19) United States
(12) Patent Application Publication
(10) Pub. No.: US 2022/0036000 A1
(43) Pub. Date: Feb. 3, 2022

(54) DEVICE-BASED COMPLIANCE BASED ON NATURAL LANGUAGE PROCESSING

(71) Applicant: International Business Machines Corporation, Tokyo (JP)

(72) Inventors: Jennifer Lynn La Rocca, Cary, NC (US); Rebecca Lynn Dahlman, Rochester, MN (US); Kristin E. McNeil, Charlotte, NC (US); Debra L. Angst, Rochester, MN (US); Mario J. Lorenzo, Miami, FL (US)

(21) Appl. No.: 16/942,050

(22) Filed: Jul. 29, 2020

(51) Int. Cl. G06F 40/279; G06N 20/00; G06F 40/242; G08B 21/24

(52) U.S. Cl. CPC G06F 40/279; G06N 20/00; G16Y 20/20; G08B 21/24; G06F 40/242

(57) ABSTRACT

Examples described herein provide a computer-implemented method that includes performing a text analysis on unstructured text to identify a restriction associated with a subject. The method further includes identifying an environmental requirement associated with the subject based at least in part on the restriction. The method further includes identifying one or more devices associated with the subject based at least in part on the environmental requirement. The method further includes receiving data from the one or more devices. The method further includes determining device usage information based at least in part on the data. The method further includes determining a compliance of the subject to the restriction based at least in part on the device usage information. The method further includes, responsive to determining non-compliance of the subject, changing a function of the one or more devices.

### BACKGROUND

Embodiments described herein generally relate to computer processing systems, and more specifically, to device-based compliance based on natural language processing.

Devices, such as Internet of Things (IoT) devices, can be sensor-enabled to collect, transmit, and/or receive data, such as data about an object or environment. The IoT is generally a network of physical objects, devices, vehicles, buildings, and other items that are embedded with electronics, software, sensors, and network connectivity, which enables these objects to collect and exchange data. These items may be in continuous communication with servers on the cloud.

### SUMMARY

Embodiments of the present invention are directed to IoT device-based compliance based on natural language processing.

A non-limiting example computer-implemented method includes Examples described herein provide a computer-implemented method that includes performing, by a text analysis engine, a text analysis on unstructured text to identify a restriction associated with a subject. The method further includes identifying, by a logic engine, an environmental requirement associated with the subject based at least in part on the restriction. The method further includes identifying, by a device engine, one or more devices associated with the subject based at least in part on the environmental requirement. The method further includes receiving, by the device engine, data from the one or more devices. The method further includes determining, by a data engine, device usage information based at least in part on the data. The method further includes determining, by a compliance engine, a compliance of the subject to the restriction based at least in part on the device usage information. The method further includes, responsive to determining non-compliance of the subject, changing, by the device engine, a function of the one or more devices.

According to one or more embodiments described herein, the text is medical text and the restriction is a medical restriction. According to one or more embodiments described herein, the text analysis is rules/dictionary based. According to one or more embodiments described herein, the text analysis is performed using machine learning. According to one or more embodiments described herein, the text analysis is performed using a hybrid of rules/dictionary based and machine learning. According to one or more embodiments described herein, the one or more devices track a usage, a duration, an activity level, an intensity, an on/off state, and metadata of the one or more devices. According to one or more embodiments described herein, the method further includes, responsive to determining non-compliance of the subject, issuing, by the device engine, an alert of the non-compliance. According to one or more embodiments described herein, the one or more devices is an Internet of Things device. According to one or more embodiments described herein, the one or more devices comprises one or more sensors to collect the data.

Other embodiments of the present invention implement features of the above-described method in computer systems and computer program products.

Advantages of the disclosed techniques include determining compliance or non-compliance with a condition or restriction that is determined from unstructured text using text analytics. When non-compliance is detected, automated actions can be practically applied to correct the non-compliance.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 depicts a block diagram of a processing system for device-based compliance based on natural language processing according to one or more embodiments described herein

FIG. 2 depicts a flow diagram of a method for device-based compliance based on natural language processing according to one or more embodiments described herein;

FIG. 3 depicts a cloud computing environment according to one or more embodiments described herein;

FIG. 4 depicts abstraction model layers according to one or more embodiments described herein; and

FIG. 5 depicts a block diagram of a processing system for implementing the presently described techniques according to one or more embodiments described herein.

The diagrams depicted herein are illustrative. There can be many variations to the diagram or the operations described therein without departing from the scope of the invention. For instance, the actions can be performed in a differing order or actions can be added, deleted or modified. Also, the term “coupled” and variations thereof describes having a communications path between two elements and does not imply a direct connection between the elements with no intervening elements/connections between them. All of these variations are considered a part of the specification.

### DETAILED DESCRIPTION

One or more embodiments of the present invention provide IoT device-based compliance based on natural language processing. Natural language processing (NLP) can be used in many contexts to analyze unstructured “natural language” text. In the medical field, healthcare providers (i.e., doctors, nurses, pharmacists, technicians, and other practitioners) often keep patient records. These patient records may be entered as unstructured text suitable for natural language processing. In these records, the healthcare provider may indicate certain restrictions, including conditions, on a patient.

Patients have various symptoms or medical conditions that require certain restricted physical or environmental behaviors. For example, a patient record may indicate that a patient should remain in low-light conditions for a certain period of time due to a particular procedure or medication associated with the patient. As another example, a patient record may indicate that a patient should walk or exercise a certain amount of time each day. A healthcare provider might suggest a change in a patient's environment, a change to how a patient uses something, a change to a patient's physical behavior, etc. However, it is difficult for the healthcare provider to monitor the patient and to know that the patient is following the healthcare provider's recommendation and staying in compliance. A patient who does not behave in a compliant way could directly and negatively impact the patient's outcome. Sometimes, the behavior that the patient needs to achieve utilizes a device, such as an IoT device.

In one conventional implementation, a sensor-enabled smart device may be recommended to a user. The smart device may have the ability to send and receive real-time data over the network. A value of the device, to the user, may be determined based on user analytics. A vendor through which to sell the device may be determined based on market analytics. A sale of the device via the vendor may be recommended to the user. However, unlike the techniques described herein, this approach does not use text analytics on medical text to determine the restriction or restriction to which the patient needs to comply.

In another conventional implementation, an integrated collaboration platform with various communication and collaboration tools includes an analytic engine to perform communication and mood analysis of conversations among other analyses. The platform enables a method for contextual communication, in an embodiment. However, unlike the techniques described herein, this conventional approach does not use text analytics on medical text to determine the restriction or requirement the patient needs to comply.

The above-described aspects of the invention address the shortcomings of the prior art by providing techniques to utilize text analytics to identify a condition or restriction associated with a subject and then using devices, such as IoT devices, to determine compliance with the condition or restriction. As an example, the present techniques use text analytics to identify conditions or restrictions described in unstructured text, such as medical records associated with a patient, and then monitors the patient using device usage information to assess compliance to the orders given by a healthcare provider.

As an example, the present techniques utilize text analytics to analyze a patient's medical unstructured text to identify medical situations or healthcare provider orders that present a restriction or requirement on a patient's environment and/or physical activity/behavior. Based on the identified medical situation for the patient, the environment and/or physical activity of the patient is identified using a medical logic engine. One or more IoT devices are linked with the medical logic engine, and the collected information from the IoT devices is fed into the system from IoT device(s). Each IoT device tracks the usage, duration, activity level, intensity, on/off, and/or other metadata of that device associated with the patient. This device information is inputted into a compliance engine, and the patient's compliance information to the medical restriction or requirement is determined. The system could alert a healthcare provider and/or the patient and/or modify the device's function when any non-compliant behavior is identified.

Consider the following example: If a patient had a brain embolism, then the doctor might indicate that the lighting in the patient's room needs to be on in order for the optimal patient outcome. If brain embolism or the lights on requirement are identified in the unstructured medical text, then once the lights are turned off in the patient's room, the system could alert the nurse or doctor of the compliance issue, automatically turn on the lights, etc.

Consider the following example: A patient might have a surgery that requires the patient's leg(s) to be elevated, or the patient has acid reflux and should sleep in an elevated position. An IoT device, such as a bed or reclining chair, could monitor the patient's usage patterns, input them into the system, and determine the patient's compliance with the requirement mentioned or inferred in the unstructured medical notes.

Example embodiments of the disclosure include or yield various technical features, technical effects, and/or improvements to technology. Example embodiments of the disclosure provide a system configured to determine compliance or non-compliance with a condition or restriction that is determined from unstructured text using text analytics. When non-compliance is detected, automated actions can be practically applied to correct the non-compliance. It should be appreciated that the above examples of technical features, technical effects, and improvements to technology of example embodiments of the disclosure are merely illustrative and not exhaustive.

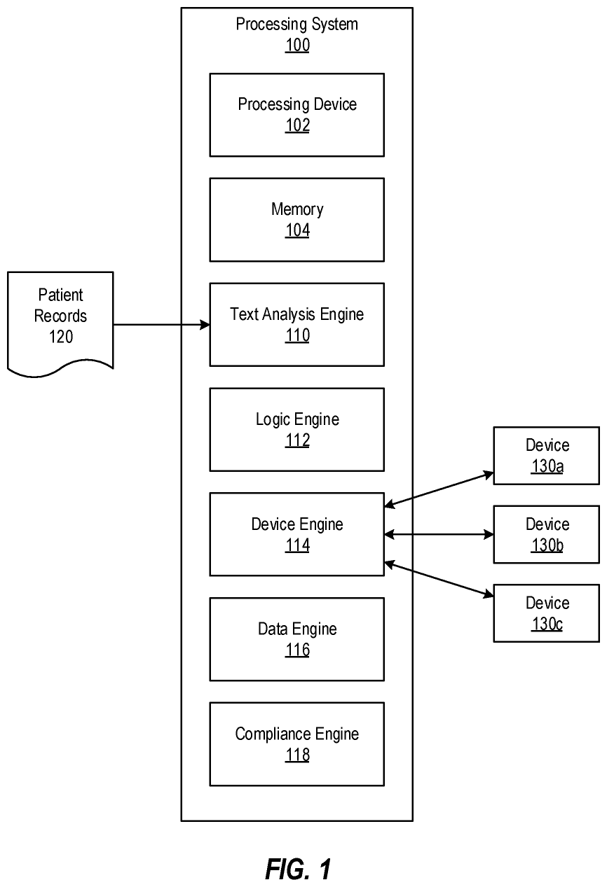

FIG. 1 depicts a block diagram of a processing system 100 for IoT device-based compliance based on natural language processing according to aspects of the present disclosure. The processing system 100 includes a processing device 102, a memory 104, a text analysis engine 110, a logic engine 112, a device engine 114, a data engine 116, and a compliance engine 118. The processing system 100 receives or collects data, such as patient records 120, and analyzes them to identify one or more conditions and/or one or more restrictions associated with a subject (e.g., a patient).

The processing system 100 can be communicatively coupled to devices 130a, 130b, 130c (collectively referred to as “devices 130”) to receive data about the subject and/or about an environment associated with the subject. Data collected by the devices 130 is then monitored to determine whether the subject is compliant or non-compliant with the condition/restriction. If non-compliance is determined, a function or functions of one or more of the devices 130 can be changed. In some examples, the devices 130 include one or more sensors or modules, such as global positioning system (GPS) modules, light sensors, temperature sensors, ultraviolet (UV) light sensors, accelerometers, gyroscopes, tilt sensors, microphones, speakers, displays, lights, etc.

The various components, modules, engines, etc. described regarding FIG. 1 can be implemented as instructions stored on a computer-readable storage medium, as hardware modules, as special-purpose hardware (e.g., application specific hardware, application specific integrated circuits (ASICs), application specific special processors (ASSPs), field programmable gate arrays (FPGAs), as embedded controllers, hardwired circuitry, etc.), or as some combination or combinations of these. According to aspects of the present disclosure, the engine(s) described herein can be a combination of hardware and programming. The programming can be processor executable instructions stored on a tangible memory, and the hardware can include the processing device 102 for executing those instructions. Thus a system memory (e.g., memory 104) can store program instructions that when executed by the processing device 102 implement the engines described herein. Other engines can also be utilized to include other features and functionality described in other examples herein.

In some examples, the devices 130 are communicatively coupled to the processing system 100 directly while in other example, the devices 130 are communicatively coupled to the processing system 100 via a network or networks (not shown). The network(s) represents any one or a combination of different types of suitable communications networks such as, for example, cable networks, public networks (e.g., the Internet), private networks, wireless networks, cellular networks, or any other suitable private and/or public networks. Further, the network(s) may have any suitable communication range associated therewith and may include, for example, global networks (e.g., the Internet), metropolitan area networks (MANs), wide area networks (WANs), local area networks (LANs), or personal area networks (PANs). In addition, the network(s) can include any type of medium over which network traffic may be carried including, but not limited to, coaxial cable, twisted-pair wire, optical fiber, a hybrid fiber coaxial (HFC) medium, microwave terrestrial transceivers, radio frequency communication mediums, satellite communication mediums, or any combination thereof.

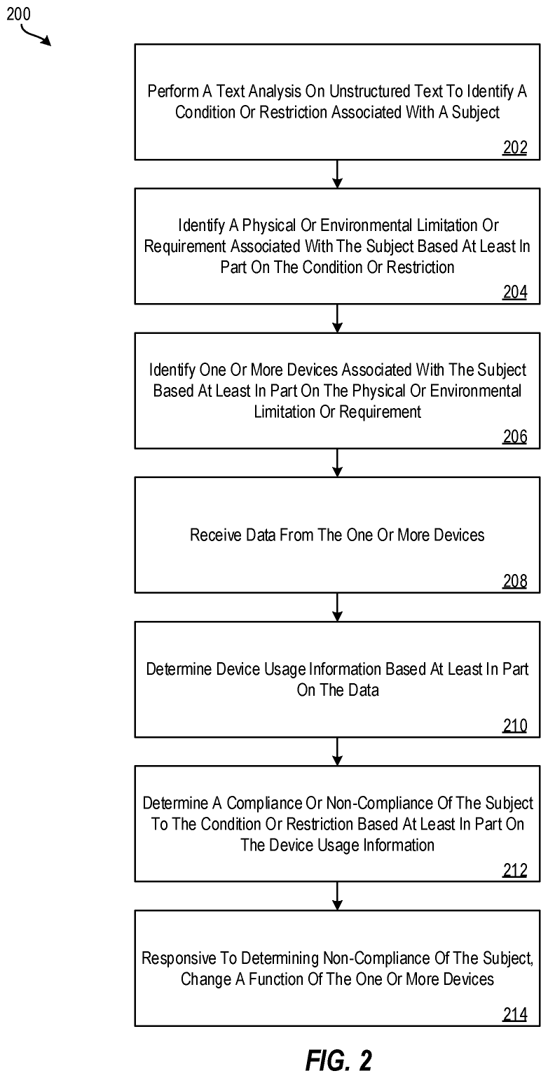

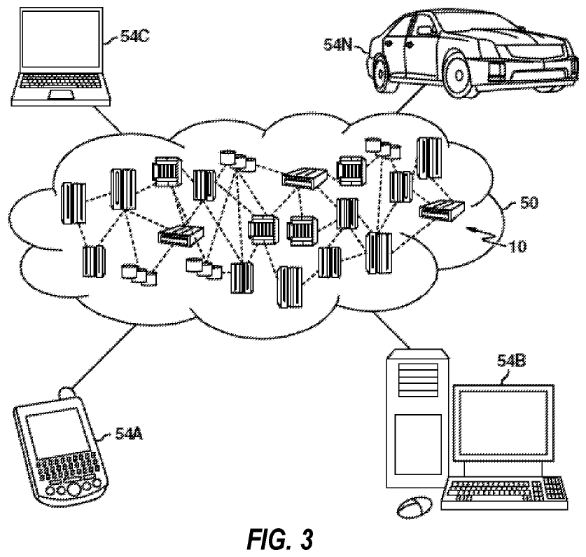

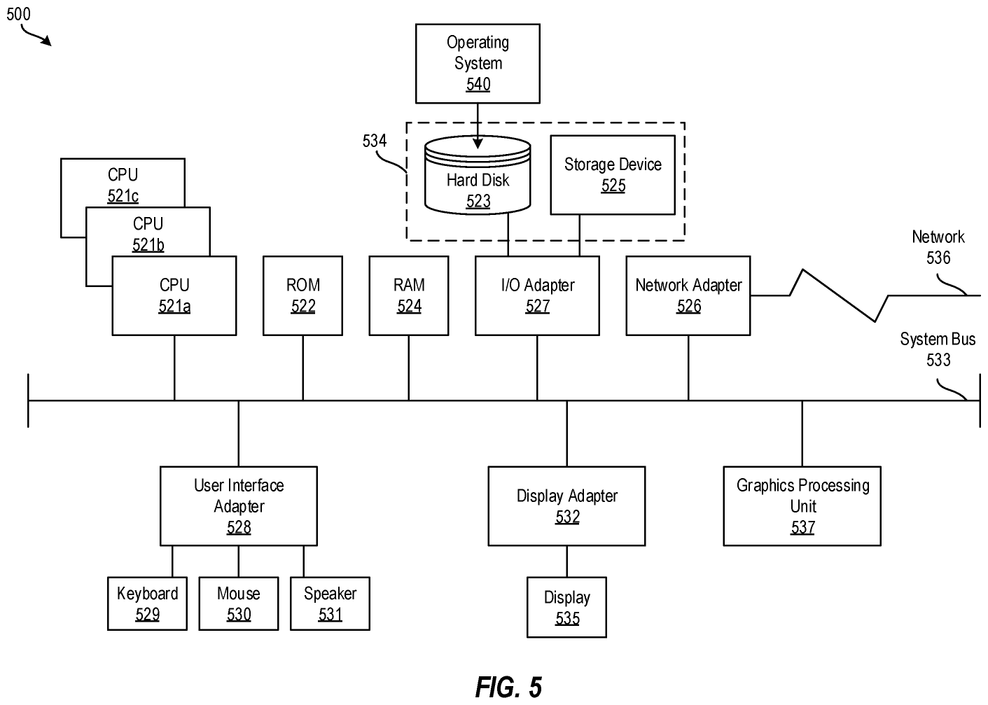

The features and functionality of the processing system 100 are now described in more detail with reference to FIG. 2. In particular, FIG. 2 depicts a flow diagram of a method 200 for device-based compliance based on natural language processing according to one or more embodiments described herein. The method 200 can be performed by any suitable processing system (e.g., the processing system 100 of FIG. 1, one or more cloud computing nodes 10 of FIG. 3, the processing system 500 of FIG. 5, etc.), by any suitable processing device (e.g., the processing device 102 of FIG. 1, the processor(s) 521 of FIG. 5, etc.), and/or suitable combinations thereof.

At block 202, the processing system 100 performs, using the text analysis engine 110, a text analysis on unstructured text to identify a condition or restriction associated with a subject. As an example, patient records 120 that are associated with a patient (i.e., a subject) are received by the processing system 100 and are processed by the text analysis engine 100. In particular, the text analysis engine 110 performs a text analysis (e.g., a rules/dictionary based text analysis, a machine learning text analysis, a hybrid of rules/dictionary based and machine learning, etc.) to identify a condition or restriction associated with the subject. According to one or more embodiments described herein, the text can be medical text, and the condition or restriction can be a medical condition or medical restriction. For example, the medical condition or medical restriction can be that patient (i.e., subject) should remain in low-light conditions for a certain period of time due to a particular medical procedure or medication associated with the patient.

At block 204, the processing system 100 identifies, using the logic engine 112, a physical or environmental limitation or requirement associated with the subject based at least in part on the condition or restriction. Continuing with the low-light example medical condition or medical restriction, the physical or environmental limitation is that light should be dimmed to 50% brightness or less or that the patient should remain indoors, for example.

At block 206, the processing system 100 identifies, using the device engine 114, one or more devices (e.g., one or more of the devices 130) associated with the subject based at least in part on the physical or environmental limitation or requirement. As an example, the device 130a can be a light switch that controls lights, the device 130b can be a smartphone associated with the patient, and the device 130c can be a wearable electronic device (e.g., a smart watch) associated with the patient. The devices 130 transmit data to and/or receive data from the processing system 100 via the device engine 114.

At block 208, the processing system 100 receives, from the device engine 114, data received from one or more of the devices 130. For example, the light switch device 130a can send data to the processing system 100 indicating current and/or historical light levels. Similarly, the smartphone device 130b and the wearable electronic device 130c can send data to the processing system 100 indicating a sensed amount of light and/or location data associated with the patient.

At block 210, the processing system 100 determines, using the data engine 116, device usage information based at least in part on the data. For example, the data engine 116 can determine, based on the data from the devices 130b and/or 130c, that the patient was outside for an extended period of time. This can be based on, for example, UV data detected by a UV sensor associated with one or more of the devices 130b, 130c, GPS data detected by a GPS module associated with one or more of the devices 130b, 130c, etc. As another example, the data engine 116 can determine, based on brightness data from the switch device 130a, that the light was set to 80% brightness. In some examples, the devices 130 sense a usage, a duration, an activity level, an intensity, an on/off state, and metadata.

At block 212, the processing system 100 determines, using the compliance engine 118, a compliance or non-compliance of the subject to the condition or restriction based at least in part on the device usage information. For example, the compliance engine 118 determines that the patient is non-compliant with the restriction to remain indoors when the data (e.g., the UV data, the GPS data, etc.) indicates that the patient was outside for an extended period of time. Similarly, the compliance engine 118 determines that the patient is non-compliant with the restriction to remain in a low-light environment when the data (e.g., the brightness data) indicates that the light was set to 80% brightness.

At block 214, the processing system 100, using the device engine 114, changes a function of one or more of the devices 130. For example, the compliance engine 118 can lower the brightness of the device 130a to 50% or less. In some examples, the compliance engine 118 can automatically lower the brightness of the light to 50% or less anytime it is set to be higher by the patient. In another example, the compliance engine 118 can issue an alert to the patient and/or to a caregiver to indicate non-compliance when detected. Such an alert can remind the patient of the condition or restriction.

Additional processes also may be included, and it should be understood that the process depicted in FIG. 2 represents an illustration, and that other processes may be added or existing processes may be removed, modified, or rearranged without departing from the scope of the present disclosure.

It is to be understood that, although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

Referring now to FIG. 3, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 3 are intended to be illustrative only and that computing nodes 10 and cloud computing environment 50 can communicate with any type of computerized device over any type of network and/or network addressable connection (e.g., using a web browser).

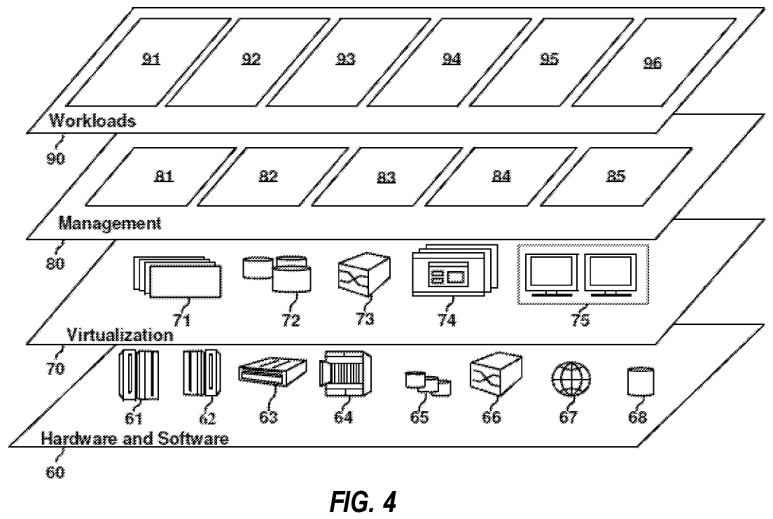

Referring now to FIG. 4, a set of functional abstraction layers provided by cloud computing environment 50 (FIG. 3) is shown. It should be understood in advance that the components, layers, and functions shown in FIG. 4 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and IoT device-based compliance based on natural language processing 96.

It is understood that one or more embodiments described herein is capable of being implemented in conjunction with any other type of computing environment now known or later developed. For example, FIG. 5 depicts a block diagram of a processing system 500 for implementing the techniques described herein. In accordance with one or more embodiments described herein, the processing system 500 is an example of a cloud computing node 10 of FIG. 3. In examples, processing system 500 has one or more central processing units (“processors” or “processing resources”) 521a, 521b, 521c, etc. (collectively or generically referred to as processor(s) 521 and/or as processing device(s)). In aspects of the present disclosure, each processor 521 can include a reduced instruction set computer (RISC) microprocessor. Processors 521 are coupled to system memory (e.g., random access memory (RAM) 524) and various other components via a system bus 533. Read only memory (ROM) 522 is coupled to system bus 533 and may include a basic input/output system (BIOS), which controls certain basic functions of processing system 500.

Further depicted are an input/output (I/O) adapter 527 and a network adapter 526 coupled to system bus 533. I/O adapter 527 may be a small computer system interface (SCSI) adapter that communicates with a hard disk 523 and/or a storage device 525 or any other similar component. I/O adapter 527, hard disk 523, and storage device 525 are collectively referred to herein as mass storage 534. Operating system 540 for execution on processing system 500 may be stored in mass storage 534. The network adapter 526 interconnects system bus 533 with an outside network 536 enabling processing system 500 to communicate with other such systems.

A display (e.g., a display monitor) 535 is connected to system bus 533 by display adapter 532, which may include a graphics adapter to improve the performance of graphics intensive applications and a video controller. In one aspect of the present disclosure, adapters 526, 527, and/or 532 may be connected to one or more I/O busses that are connected to system bus 533 via an intermediate bus bridge (not shown). Suitable I/O buses for connecting peripheral devices such as hard disk controllers, network adapters, and graphics adapters typically include common protocols, such as the Peripheral Component Interconnect (PCI). Additional input/output devices are shown as connected to system bus 533 via user interface adapter 528 and display adapter 532. A keyboard 529, mouse 530, and speaker 531 may be interconnected to system bus 533 via user interface adapter 528, which may include, for example, a Super I/O chip integrating multiple device adapters into a single integrated circuit.

In some aspects of the present disclosure, processing system 500 includes a graphics processing unit 537. Graphics processing unit 537 is a specialized electronic circuit designed to manipulate and alter memory to accelerate the creation of images in a frame buffer intended for output to a display. In general, graphics processing unit 537 is very efficient at manipulating computer graphics and image processing, and has a highly parallel structure that makes it more effective than general-purpose CPUs for algorithms where processing of large blocks of data is done in parallel.

Thus, as configured herein, processing system 500 includes processing capability in the form of processors 521, storage capability including system memory (e.g., RAM 524), and mass storage 534, input means such as keyboard 529 and mouse 530, and output capability including speaker 531 and display 535. In some aspects of the present disclosure, a portion of system memory (e.g., RAM 524) and mass storage 534 collectively store the operating system 540 such as the AIX® operating system from IBM Corporation to coordinate the functions of the various components shown in processing system 500.

Various embodiments of the invention are described herein with reference to the related drawings. Alternative embodiments of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” may be understood to include any integer number greater than or equal to one, i.e. one, two, three, four, etc. The terms “a plurality” may be understood to include any integer number greater than or equal to two, i.e. two, three, four, five, etc. The term “connection” may include both an indirect “connection” and a direct “connection.”

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instruction by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A computer-implemented method comprising:
performing, by a text analysis engine, a text analysis on unstructured text to identify a restriction associated with a subject;
identifying, by a logic engine, an environmental requirement associated with the subject based at least in part on the restriction;
identifying, by a device engine, one or more devices associated with the subject based at least in part on the environmental requirement;
receiving, by the device engine, data from the one or more devices;
determining, by a data engine, device usage information based at least in part on the data;
determining, by a compliance engine, a compliance of the subject to the restriction based at least in part on the device usage information; and
responsive to determining non-compliance of the subject, changing, by the device engine, a function of the one or more devices.

2. The computer-implemented method of claim 1, wherein the text is medical text, and wherein the restriction a medical restriction.

3. The computer-implemented method of claim 1, wherein the text analysis is rules/dictionary based.

4. The computer-implemented method of claim 1, wherein the text analysis is performed using machine learning.

5. The computer-implemented method of claim 1, wherein the text analysis is performed using a hybrid of rules/dictionary based and machine learning

6. The computer-implemented method of claim 1, wherein the one or more devices track a usage, a duration, an activity level, an intensity, an on/off state, and metadata of the one or more devices.

7. The computer-implemented method of claim 1, further comprising:
responsive to determining non-compliance of the subject, issuing, by the device engine, an alert of the non-compliance.

8. The computer-implemented method of claim 1, wherein the one or more devices is an Internet of Things device.

9. The computer-implemented method of claim 1, wherein the one or more devices comprises one or more sensors to collect the data.

10. A system comprising:
a memory comprising computer readable instructions; and
a processing device for executing the computer readable instructions, the computer readable instructions controlling the processing device to perform operations comprising:
performing, by a text analysis engine, a text analysis on unstructured text to identify a restriction associated with a subject;
identifying, by a logic engine, an environmental requirement associated with the subject based at least in part on the restriction;
identifying, by a device engine, one or more devices associated with the subject based at least in part on the environmental requirement;
receiving, by the device engine, data from the one or more devices;
determining, by a data engine, device usage information based at least in part on the data;
determining, by a compliance engine, a compliance of the subject to the restriction based at least in part on the device usage information; and
responsive to determining non-compliance of the subject, changing, by the device engine, a function of the one or more devices.

11. The system of claim 10, wherein the text is medical text, and wherein the restriction a medical restriction.

12. The system of claim 10, wherein the text analysis is rules/dictionary based.

13. The system of claim 10, wherein the text analysis is performed using machine learning.

14. The system of claim 10, wherein the text analysis is performed using a hybrid of rules/dictionary based and machine learning

15. The system of claim 10, wherein the one or more devices track a usage, a duration, an activity level, an intensity, an on/off state, and metadata of the one or more devices.

16. The system of claim 10, wherein the operations further comprise:
responsive to determining non-compliance of the subject, issuing, by the device engine, an alert of the non-compliance.

17. The system of claim 10, wherein the one or more devices is an Internet of Things device.

18. The system of claim 10, wherein the one or more devices comprises one or more sensors to collect the data.

19. A computer program product comprising a computer readable storage medium having program instructions embodied therewith, the program instructions executable by a processor to cause the processor to perform operations comprising:
performing, by a text analysis engine, a text analysis on unstructured text to identify a restriction associated with a subject;
identifying, by a logic engine, an environmental requirement associated with the subject based at least in part on the restriction;
identifying, by a device engine, one or more devices associated with the subject based at least in part on the environmental requirement;
receiving, by the device engine, data from the one or more devices;
determining, by a data engine, device usage information based at least in part on the data;
determining, by a compliance engine, a compliance of the subject to the restriction based at least in part on the device usage information; and
responsive to determining non-compliance of the subject, changing, by the device engine, a function of the one or more devices.

20. The computer program product of claim 19, wherein the text is medical text, and wherein the restriction a medical restriction.
